(19) United States
(12) Patent Application Publication
(10) Pub. No.: US 2003/0224050 A1
(43) Pub. Date: Dec. 4, 2003

(54) Drug delivery system for sustained delivery of glipizide

(76) Inventors: Charles Chiao, Parkland, FL (US); Ping He, Miami, FL (US)

Correspondence Address: Dennis A. Emma, Ph.D., 4400 Biscayne Boulevard, Miami, FL, 33137, US

(21) Appl. No.: 10/336,243

(22) Filed: Jan. 4, 2003

Related U.S. Application Data
(60) Provisional application No. 60/345,267, filed on Jan. 4, 2002.

(51) Int. Cl. A61K 31/426; A61K 9/24

(52) U.S. Cl. 424/471; 514/369; 514/57

(57) ABSTRACT

Disclosed are compositions and methods of use for the sustained release of glipizide reducing the drug dumping associated with conventional approaches. Such compositions include glipizide and a carbomer admixed to form a core substantially coated with a film coat.

This application claims priority to U.S. Provisional Application Ser. No.60/345,267 filed on Jan. 4, 2002.

### BACKGROUND OF THE INVENTION

1. Field of the Invention

The invention relates to formulations, particularly formulations allowing the sustained delivery of an active component in the formulation, such as glipizide.

2. Summary of the Related Art

When compared to conventional immediate release formulations, sustained drug delivery systems have the advantages of reduced fluctuations in the blood level of drug as well as a reduction in the dosing frequency ultimately enhancing patient convenience, compliance, and lowering overall health care costs.

U.S. Pat. No. 5,945,125 describes a drug release system based on swelling/erosion, where the release of the active substance from a single homogenous mixture occurs at a mathematically defined rate. The drug release system is an uncoated tablet containing a water-swellable, rate controlling polymer polyethylene oxide) and a lubricant, where the swellable polymer is chosen such that its rate of swelling is equal to its dissolution rate. This supposedly creates a tablet that retains its initial size throughout drug delivery so that the tablet is fully dissolved at the time that the last of the drug has been released. The patent does not teach the use of pH-dependent and/or pH-independent coat polymers. Additionally, the reference does not describe the use of carbomer (a homopolymer of acrylic acid cross linked with an allyl ether of pentaeyrhritol, an allyl ether of sucrose or an allyl ether of propylene) as a rate controlling polymer for the tablet core.

Some sustained drug delivery systems include pharmaceutically acceptable polymers such as hydroxypropyl methylcellulose (HPMC) and polyethylene oxide (see, e.g., Pham and Lee, Pharmaceutical Research 11: 1379-1384, 1994; Apicella et al., Biomaterials 24: 83-90, 1993; Colombo et. al., J. Pharm Sci. 84: 991-997, 1995; Gao et al., Pharmaceutical Research 12: 965-971, 1995).

U.S. Pat. No. 6,048,547 describes a delayed release pharmaceutical composition containing at least 22% of an active ingredient and polyethylene oxide. However, this reference does not teach the use of carbomer or pH-dependent or pH-independent coat polymers as a means for additionally controlling the release of the pharmaceutical agent.

One drawback of the above-described systems is that often, there is a burst-effect, releasing a substantial portion of the active ingredient at one time. Some systems, such as that described in U.S. Pat. No. 5,885,616, are designed to have an immediate release of a substantial portion of the active ingredient followed by the sustained release of the remaining portion of the active ingredient.

Thus, there remains a need for a pharmaceutical composition that allows the sustained release of an active ingredient, such as glipizide, while also minimizing the burst-effect of the active ingredient.

### SUMMARY OF THE INVENTION

The invention provides formulations for the sustained release of an active ingredient, particularly glipizide. The substantial uniformity of the release of the active ingredient according to the invention minimizes the burst-effect during the initial dissolution of the active ingredient observed in the prior art.

The invention provides compositions and methods of use of the same for the sustained delivery of glipizide, comprising glipizide and a carbomer admixed to form a core substantially coated with a film coat comprising a film coat polymer.

In an embodiment of the invention, the composition further includes at least one core polymer. Core polymers contemplated herein include hydroxypropyl methylcellulose and polyethylene oxide.

The invention further comprises a film coat including at least one film coat polymer which is a pH-dependent or pH-independent polymer or a combinations thereof. The pH-dependent polymers contemplated are methacrylic acid/methyl methacrylate copolymer, methacrylic acid/ethyl acrylate copolymer, methacrylic acid/ methyl acrylate and methyl methacrylate copolymer, cellulose acetate phthalate, cellulose acetate trimellitate, hydroxypropyl methylcellulose acetate succinate, hydroxypropyl methylcellulose phthalate, polyvinyl acetate phthalate, methacrylic acid copolymer type C, methacrylic acid copolymer type A, polyacrylate dispersion, and shellac. The pH-independent polymers contemplated include ethylcellulose, methacrylic ester copolymer, and ammonio methacrylate copolymer.

An embodiment of the invention includes about 94% of a core and about 6% of a film coat polymer by weight of the composition, wherein the core comprises about 2.5% of glipizide, about 15% of polyethylene oxide, and about 31% of hydroxypropyl methylcellulose by weight of the core, and the coat polymer includes about 4% of methacrylic acid/methyl methacrylate copolymers and methacrylic ester copolymer by weight of the composition. This composition further includes a film coat containing about 0.8% triethyl citrate by weight of the composition.

Another embodiment includes about 94% of a core and about 6.0% of a film coat by weight of the composition, the core including about 2.5% of glipizide, about 14% of carbomer by weight of the core, and the film coat polymer including about 4% of methacrylic acid copolymers by weight of the composition. This composition may also include a film coat containing about 0.8% triethyl citrate by weight of the composition.

In yet another embodiment, the composition of the invention includes about 95.7% of a core and about 4.3% of a film coat by weight of the composition, the core including about 2.5% of glipizide, about 14% of hydroxypropyl methylcellulose, and about 6% of carbomer by weight of the core, and the coat polymer including about 3% of methacryiic acid copolymers by weight of the composition. This composition may additionally include a film coat containing about 0.6% triethyl citrate by weight of the composition.

Another embodiment includes about 96.3% of a core and about 3.7% of a film coat by weight of the composition, the core including about 2.5% of glipizide and about 15% of a hydroxypropyl methylcellulose by weight of the core, and the film coat including about 3% of ethyl cellulose by weight of the composition. The composition may additionally include a film coat containing about 0.7% triethyl citrate by weight of the composition.

Another embodiment includes about 92.1% of a core and about 7.9% of a film coat by weight of the composition, the core including about 2.5% of glipizide, about 14% of hydroxypropyl methylcellulose, and about 6% of carbomer by weight of the core, and the film coat polymer including about 5% of methacrylic acid copolymers by weight of the composition. This composition further may additionally include a film coat containing about 0.6% triethyl citrate by weight of the composition.

An aspect of the invention discloses pharmaceutical compositions and methods of using the same for the sustained delivery of glipizide for use in the treatment of glipizide responsive disorders. In this aspect, the compositions comprise a therapeutically effective amount of glipizide admixed with carbomer and/or core polymers to form a core. The core provided is substantially coated with a rate controlling film coat comprising at least one film coat polymer. Additional pharmaceutically acceptable ingredients may be incorporated into the core and/or film coat.

### BRIEF DESCRIPTION OF THE DRAWINGS

The foregoing and other objects of the present invention, the various features thereof, as well as the invention itself may be more fully understood from the following description, when read together with the accompanying drawings in which:

FIG. 1 is a representation of a line graph showing the in vitro dissolution of glipizide in a buffer of pH 7.5 from Formula A, a non-limiting, representative formulation of the invention, where the formulation is uncoated (diamonds) or coated (squares).

FIG. 2 is a representation of a line graph showing the in vitro dissolution of glipizide in a buffer of pH 7.5 from Formula B, a non-limiting, representative formulation of the invention, where the formulation is uncoated (diamonds) or coated (squares).

FIG. 3 is a representation of a line graph showing the dissolution rate from Formula B tablet cores and coated tablets plotted as log t vs. log (Mₜ/M_{T}), where t is time, Mₜ/M_{T} is the fraction of the pharmaceutical agent that has been released at time t from the tablet.

FIG. 4 is a representation of a line graph showing the plasma concentration of glipizide from coated glipizide sustained release tablets for Formula A (diamonds) or Formula B (squares) formulations in a crossover study of 11 fasting healthy volunteers.

### DETAILED DESCRIPTION

The published patent and scientific literature referred to herein establishes knowledge that is available to those with skill in the art. The issued U.S. patents, allowed applications, published foreign patent applications, and references that are cited herein are hereby incorporated by reference to the same extent as if each was specifically and individually indicated to be incorporated by reference. Any inconsistency between these publications and the present disclosure shall be resolved in favor of the present disclosure.

As used in this specification, the singular forms “a,” “an” and “the” specifically also encompass the plural forms of the terms to which they refer, unless the content clearly dictates otherwise. For example, reference to “an active drug” includes mixtures of active drugs.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, unless specifically indicated otherwise, the word “or” is used in the “inclusive” sense of “and/or” and not the “exclusive” sense of “either/or.”

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

The technical and scientific terms as used herein have the meaning commonly understood by one of skill in the art to which the present invention pertains, unless defined otherwise. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Standard reference works setting forth the general principles of pharmaceutical formulations include Remington's Pharmaceutical Sciences, 18^{th} Ed., Gennaro, Mack Publishing Co., Easton, Pa. (1990) and Remington: The Science and Practice of Pharmacy, Lippincott, Williams & Wilkins (1995).

The invention stems from the development of new formulations that allow for the sustained, uniform release of an active drug substance, such as glipizide. The formulations of the invention are based on a matrix system, which is composed of high molecular weight polymers that allow the active drug substance to be released over an extended period.

As used herein, “sustained release” or “sustained delivery” denotes a pharmacokinetic profile wherein the therapeutically effective amount of the medicament is released from the formulation at a controlled rate such that therapeutically beneficial blood levels (but below toxic levels) of the active ingredient are maintained over an extended period of time, e.g., providing a 24 hour dosage form. The advantages include less frequent dosing of a medicament and resultant patient compliance, a more sustained drug blood level response, therapeutic action with less ingested drug and possibly, the mitigation of side effects. By providing a slow and steady release of the medicament over time, absorbed concentration spikes are mitigated or even eliminated by effecting a smoother and more sustained blood level response.

In accordance with the invention, included among “active drug” substances are, without limitation, glipizide, carbamazepine, simvastatin, nifedipine, omeprazole, venlafaxine hydrochloride, rabeprazole sodium, paroxetine hydrochloride, and amlodipine besylate. The exemplified active drug substance of the invention is glipizide.

One drawback of known matrix systems is that there is an initial burst effect at the initiation of dissolution, such that a large initial dosage of the active drug substance is released or “dumped” from the system. No drug dumping was observed in the optimized systems of the present invention even from an in vivo clinical study. According to the invention, the sustained-release composition comprises a tablet core that is composed of polymeric material, an active drug substance, and pharmaceutically acceptable inactive or active ingredients, where the active drug substance is distributed uniformly in the core. The systems of the invention are akin to a monolithic matrix system, and the dissolution profile of the active drug substance release from the material approximates a zero-order (or close to zero-order) dissolution. The core is coated with, for example, a pharmaceutically acceptable coating material.

The systems of the invention have the characteristics of a combination of monolithic and reservoir systems. Thus, the systems of the invention provide not only sustained release of the active drug substance, but also uniform release of the active substance with a reduced drug dumping effect.

The invention provides compositions for the sustained delivery of glipizide, comprising glipizide and a carbomer admixed to form a core substantially coated with a film coat comprising a film coat polymer.

As used herein, “carbomer” means a homopolymer of acrylic acid cross linked with either an allyl ether of pentaeythritol, an allyl ether of sucrose or an allyl ether of propylene (e.g., available from B. F. Goodrich, Cleveland, Ohio, USA).

Contemplated compositions include from about 0.1% to about 15% by weight of the core of glipizide. In some other compositions from about 1% to about 5% by weight of the core of glipizide, while in yet other compositions include about 2.5% by weight of the core of glipizide.

Compositions of the invention contain from about 2% to about 30% by weight of the core of carbomer. Some compositions of the invention contain about 6.0% by weight of the core of carbomer.

In an embodiment, the compositions include from about 0.1% to about 15% by weight of the core of glipizide, and from about 2% to about 30% by weight of the core of carbomer.

As used herein, “core polymer” means polymers which, when incorporated into the composition core, serve to retard the dissolution of the core. Examples of core polymers include, without limitation, hydroxypropyl methylcellulose HPMC (e.g., with a viscosity of 100 cps to 100,000 cps available from Dow Chemical Co., Midland, Mich., USA), polyethylene oxide (e.g., with a molecular weight of 100,000 to 7,000,000 available from Union Carbide Corp., Nitro, W.Va., USA), carbomer, alginate (International Specialty Products, San Diego, Calif., USA), and chitosan.

In yet other embodiments of the invention, the compositions include about 2.5% by weight of the core of glipizide, and about 6% by weight of the core of carbomer.

The compositions may therefore further include at least one additional core polymer in a range from about 5% to about 50% by weight of the core of hydroxypropyl methylcellulose or from about 10% to about 30% by weight of the core of hydroxypropyl methylcellulose. Some compositions include from about 14% to about 15% by weight of the core of hydroxypropyl methylcellulose. Yet other compositions include about 31% by weight of the core of hydroxypropyl methylcellulose.

In another embodiment of the invention, the composition includes from about 10% to about 30% by weight of the core of polyethylene oxide. In another embodiment, the composition includes about 15% by weight of the core of polyethylene oxide.

One embodiment of the invention includes a composition of about 15% by weight of the core of hydroxypropyl methylcellulose and about 15% by weight of the core of polyethylene oxide.

As used here, “coat polymer” is meant a polymer which, when incorporated into the film coat surrounding the composition core, serves to retard the dissolution of the core. Examples of coat polmers include, for example, pH-dependent polymer(s) or pH-independent polymer(s).

Contemplated pH-dependent polymers are methacrylic acid/methyl methacrylate copolymer (see e.g., EUDRAGIT L 100, EUDRAGIT S 100 available from Rohm GmbH Pharma Polymers, Darmstadt, Del.), methacrylic acid/ethyl acrylate copolymer (see e.g., EUDRAGIT L 30D-55 available from Rohm GmbH Pharma Polymers, Darmstadt, Del.), methacrylic acid/ methyl acrylate and methyl methacrylate copolymer (e.g., EUDRAGIT FS 30D available from Rohm GmbH Pharma Polymers, Darmstadt, Del.), cellulose acetate phthalate, cellulose acetate trimellitate, hydroxypropyl methylcellulose acetate succinate, hydroxypropyl methylcellulose phthalate, polyvinyl acetate phthalate, methacrylic acid copolymer type C, methacrylic acid copolymer type A, polyacrylate dispersion, and shellac. pH-independent polymers contemplated are ethylcellulose(available from FMC Corp., Philadelphia, Pa., USA), methacrylic ester copolymer (e.g., EUDRAGIT NE30D available from Rohm GmbH Pharma Polymers, Darmstadt, Del.), and ammonio methacrylate copolymer (e.g., EUDRAGIT RL 30D and EUDRAGIT RS 30D available from Rohm GmbH Pharma Polymers, Darmstadt, Del.).

The compositions may further include from about 1% to about 20% by weight of the core of a film coat polymer. Other embodiments may include from about 2% to about 10% or from about 3% to about 5% by weight of the core of a film coat polymer. Yet other embodiments of the invention, the compositions include a total of about 3% by weight of the core of one or more film coat polymers, e.g., methacrylic acid copolymer type A, methacrylic acid copolymer type C, ethylcellulose, and polyacrylate dispersion.

An embodiment of the invention includes about 94% of a core and about 6% of a film coat polymer by weight of the composition, wherein the core includes about 2.5% of glipizide, about 15% of polyethylene oxide, and about 31% of hydroxypropyl methylcellulose by weight of the core, and the coat polymer including about 4% of methacrylic acid/methyl methacrylate copolymers and methacrylic ester copolymer by weight of the composition. This composition further includes a film coat containing about 0.8% triethyl citrate (e.g., Morflex, Inc., Greensboro, N.C,, USA) by weight of the composition.

Another embodiment includes about 94% of a core and about 6.0% of a film coat by weight of the composition, the core including about 2.5% of glipizide, about 14% of carbomer by weight of the core, and the film coat polymer including about 4% of methacrylic acid copolymers (e.g., EUDRAGIT NE 30 D and EUDRAGIT L30 D-55) by weight of the composition. This composition may further include a film coat containing about 0.8% triethyl citrate by weight of the composition.

In yet another embodiment, the composition for the sustained delivery of glipizide includes about 95.7% of a core and about 4.3% of a film coat by weight of the composition, the core including about 2.5% of glipizide, about 14% of hydroxypropyl methylcellulose, and about 6% of carbomer by weight of the core, and the coat polymer including about 3% of methacrylic acid copolymers (e.g., EUDRAGIT L 100) by weight of the composition. This composition may further comprise a film coat containing about 0.6% triethyl citrate by weight of the composition.

Another contemplated embodiment describes a composition including about 96.3% of a core and about 3.7% of a film coat by weight of the composition, the core including about 2.5% of glipizide and about 15% of a hydroxypropyl methylcellulose by weight of the core, and the film coat including about 3% of ethyl cellulose by weight of the composition. The composition further includes a film coat containing about 0.7% triethyl citrate by weight of the composition.

Another envisioned embodiment discloses a composition comprising about 92.1% of a core and about 7.9% of a film coat by weight of the composition, the core comprising about 2.5% of glipizide, about 14% of hydroxypropyl methylcellulose, and about 6% of carbomer by weight of the core, and the film coat polymer comprising about 5% of methacrylic acid copolymers (e.g., EUDRAGIT L 30 D-55) by weight of the composition. This composition may further comprise a film coat containing about 0.6% triethyl citrate by weight of the composition.

A composition according to the present invention maybe a tablet. As used in accordance with the invention, by “tablet” is meant a compressed or molded small mass of material containing at least one active drug substance with or without at least one pharmaceutically acceptable inactive ingredient. While the invention is described as a tablet, one of skill in the art would know other configurations which are capable of generating the same results as described herein without undue experimentation. Thus, the compositions of the invention may be formulated to take various forms including caplets, capsules, pellets, granules, and beads. Hence, in some embodiments the compositions according to the invention are prepared as a single tablet including a therapeutically effective amount of the active ingredient whereas in other embodiments the compositions are prepared as a plurality of smaller units (e.g., pellets) to be administered as part of a single unit dosage form.

Standard methods (e.g., direct compression or granulation) for forming tablets are known to those of skill in the art. For example, the ingredients of the tablet core of a formulation of the invention can be blended together in a mixer or blender. This blend may be passed through a miller, and then pressed into tablets using a standard tablet press. The tablet is then coated with the film coat according to conventional methods including, for example, a fluid bed coating unit or an Accela-Cota or Compu-Lab coating pan. Additionally, for example, the film-coating polymer (see formulations infra) can be sprayed onto the tablet core in amounts pre-determined to substantially coat the tablet core.

As mentioned hereinabove, additional conventional pharmaceutically acceptable ingredients may also be included in the compositions of the invention. In accordance with the invention, the term “pharmaceutically acceptable” or “pharmaceutically acceptable ingredient” refers to any material, solvent, dispersion media, coating, carrier, diluent, excipient, glidant (silicon dioxide, e.g., SYLOID available from W. R. Grace Co., Baltimore, Md., USA), surfactant (e.g., TWEEN 80 available from Sigma-Aldrich Corp. St. Louis, Mo., USA) filler (such as acacia or lactose, commercially available from TIC Gums, Belcamp, Md., USA), lubricant (e.g., magnesium stearate available from Sigma-Aldrich Corp. St. Louis, Mo., USA) antifoaming agent, colorants (e.g., OPADRY II White available from available from Sigma-Aldrich Corp. St. Louis, Mo., USA), flavorings or any other pharmaceutical excipient that is non-toxic to an animal (e.g., a human) to which the pharmaceutically acceptable ingredient is administered. Preferably, the pharmaceutically acceptable ingredient of the invention does not reduce the therapeutic activity of the active drug substance with which it accompanies. Except insofar as any conventional ingredient is incompatible with the active drug substance, its use as a pharmaceutically acceptable ingredient in the tablets of the invention is contemplated.

The invention thus discloses pharmaceutical compositions and methods of using the same for the sustained delivery of an active ingredient as described above for the first aspect of the invention. The sustained release of glipizide for use in the treatment of glipizide responsive disorders is especially contemplated. Accordingly, the pharmaceutical compositions comprise a therapeutically effective amount of glipizide admixed with carbomer and/or core polymers to form a core. The core provided is substantially coated with a rate controlling film coat comprising at least one film coat polymer. Additional pharmaceutically acceptable ingredients may be incorporated e.g., into the core and/or film coat.

The term “therapeutically effective amount” is used to denote treatments at dosages effective to achieve the therapeutic result sought which is well known in the art (see e.g., Goodman and Gilman's The Pharmacological Basis of Therapeutics, 10^{th} Ed., McGraw Hill Companies, Inc., New York, pages 1701-1704 (2001)). Furthermore, one of skill will appreciate that the therapeutically effective amount of the active ingredient administered using the compositions of the invention maybe lowered or increased by fine tuning and/or by administering a composition of the invention with another active ingredient.

The invention further provides methods for treating a mammal by the administration of a therapeutically effective amount of a composition according to the invention. The methods of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

In a non-limiting example of the invention, the tablet core and film coats contain the following ingredients listed in Table I at the indicated percentages by weight.

TABLE I
Glipizide Sustained-Release Tablets
Tablet Cores

| | | Range % | Exam- | |
|---|---|---|---|---|
| Ingredient | Grade | (w/w) | ple % | Function |
| Glipizide | M.W. 445.55 | 1-5 | 2.5 | Active drug |
| Hydroxy- | Viscosity | 10-40 | 15.0 | Rate controlling |
| propyl | 4,000-15,000 cps | | | polymer |
| Methyl- | | | | |
| cellulose | | | | |
| Lactose | | 10-80 | 50.2 | Filler |
| Acacia | | 0-60 | 31.4 | Filler (& binder) |
| Silicon | | 0.1-2 | 0.3 | Glidant |
| Dioxide | | | | |
| Magnesium | | 0.2-3 | 0.6 | Lubricant |
| Stearate | | | | |
| Total | | | 100.0 | |

Film Coat

| | Range of solid | | |
|---|---|---|---|
| Ingredients | content % (w/w) | Example % | Function |
| Tablet Cores | 85-98 | 96.3 | |
| Ethyl Cellulose | 2-15 | 3.0 | Coating polymer |
| Triethyl Citrate | 0.1-5 | 0.7 | Plasticizer |
| Total | | 100.0 | |

In another non-limiting example of the invention, the tablet core and film coats contain the following ingredients listed in Table II at the indicated percentages by weight.

TABLE II
Glipizide Extended-release Tablets

| | | | | |
|---|---|---|---|---|
| | | Range % | Exam- | |
| Ingredient | Grade | (w/w) | ple % | Function |

Tablet Cores

| | | | | |
|---|---|---|---|---|
| Glipizide | | 1-5 | 2.5 | Active drug |
| Carbomer | 934P NF | 2-30 | 6.0 | Rate controlling |
| | | | | polymer |
| Hydroxy- | Viscosity | 5-40 | 14.0 | Rate controlling |
| propyl | 4,000-15,000 cps | | | polymer |
| Methyl- | | | | |
| cellulose | | | | |
| Acacia | | 0-50 | 35.0 | Filler (& binder) |
| Lactose | | 0-70 | 41.5 | Filler |
| Silicon | | 0.1-2 | 0.4 | Glidant |
| Dioxide | | | | |
| Magnesium | | 0.2-3 | 0.6 | Lubricant |
| Stearate | | | | |
| Total | | | 100.0 | |

Coat Formula

| | | | |
|---|---|---|---|
| Tablet Cores | 85-98 | 95.7 | |
| EUDRAGIT | 2-15 | 3.0 | Coating polymer |
| L 100 | | | |
| Triethyl | 0.1-5 | 0.6 | Plasticizer |
| Citrate | | | |
| Talc | 0-10 | 0.7 | Glidant |
| Total | | 100.0 | |

In certain embodiments of the invention, the core comprises between about 6% and about 31% of one or more core polymers by weight of the core, where the core polymer is either hydroxypropyl methylcellulose or polyethylene oxide. In some embodiments, the core of the tablet comprises about 31% of hydroxypropyl methylcellulose and about 15% of polyethylene oxide by weight of the core.

Thus, in a non-limiting example of the invention, the tablet core and film coats contain the following ingredients listed in Table III at the indicated percentages by weight.

TABLE III
Glipizide Sustained-Release Tablets

| | | | | |
|---|---|---|---|---|
| | | Range % | Exam- | |
| Ingredient | Grade | (w/w) | ple % | Function |

Tablet Cores

| | | | | |
|---|---|---|---|---|
| Glipizide | | 1-5 | 2.5 | Active drug |
| Polyethylene | M.W. 2,000,000 | 5-40 | 15.0 | Rate controlling |
| Oxide | | | | polymer |
| Hydroxy- | Viscosity | 0-50 | 31.0 | Rate controlling |
| propyl | 3-6 cps | | | polymer |
| Methyl- | | | | |
| cellulose | | | | |
| Lactose | | 10-80 | 50.0 | Filler (& binder) |
| Silicon | | 0.1-2 | 0.3 | Glidant |
| Dioxide | | | | |
| Magnesium | | 0.2-3 | 1.2 | Lubricant |
| Stearate | | | | |
| Total | | | 100.0 | |

Coat Formula

| | | | |
|---|---|---|---|
| Tablet Cores | 85-98 | 94.0 | |
| EUDRAGIT | 1-15 | 4.00 | Coating |
| NE 30 | | | polymer |
| EUDRAGIT | | | |
| L 30 D-55 | | | |
| Triethyl | 0.2-5 | 0.80 | Plasticizer |
| Citrate | | | |
| Glyceryl | 0.02-0.5 | 0.12 | Glidant |
| Monostea- | | | |
| rate | | | |
| TWEEN 80 | 0-0.1 | 0.025 | Surfactant |
| Simethicone | 0-0.01 | 0.005 | Antifoaming |
| | | | agent |
| Colorant | 0-5 | 1.05 | Colorant |
| (e.g. | | | |
| OPADRY II | | | |
| White) | | | |
| Total | | 100.00 | |

The invention additionally provides a means for the development of newer sustained delivery formulations of, for example, glipizide which would have clinically desirable drug release kinetic profiles. By way of example, the drug release kinetic parameters for a formulation comprising glipizide and a core polymer may be calculated, wherein glipizide is released from the tablet at a rate expressed as Mₜ/M_{T}=ktⁿ where: t is time, Mₜ/M_{T} is the fraction of the pharmaceutical agent, here glipizide, which has been released at time t, k is a constant, and n is the release kinetics exponent.

To calculate the “n” values for the formulations of the invention, the logarithmic form of the above equation was first determined. Thus, Log (Mₜ/M_{T})=Log k+n·Log t.

Using this equation, dissolution data from a formulation of the invention may be plotted Log t vs. log Mₜ/M_{T} values. The slope of the line is the “n” value. It should be noted that although the plot of log t vs. log Mₜ/M_{T} may give rise to a straight line that eventually curves with increasing log t values, the “n” is determined from the straight line normally using the dissolution data (≧70%).

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLES

### Example I

### Formula A

In a non-limiting formulation of the invention, Formula A, the following ingredients as described in Table III were used in the tablet core and for the film coat.

To prepare the tablet core of Formula A, all of the ingredients (excepting magnesium stearate) listed in Table III were blended together in a PK blender. Fifty percent (50%) of the magnesium stearate (e.g., Mallinckrodt, St. Louis, Mo., USA) was then added to the mixture, which was subsequently blended again. After the second blending, the blended mixture was compressed into slugs, which are then passed through a miller. The remaining magnesium stearate is added to the milled granules and blended again to form the final core blend. The milled final core blend was then compressed into tablets.

One of skill in the art will appreciate that alternative methods to assemble a tablet core are well known in the art and could be used instead of the steps described supra without altering the invention or its effectiveness.

The tablet cores, after formation, were next coated with a film coat having the formula described in Table III. To coat the tablet core with the indicated amount of film coat, a EUDRAGIT NE 30D dispersion is added to a ELDRAGIT L 30D 55 dispersion to form the ELDRAGIT dispersion. Separately, a glyceryl monostearate (GMS) dispersion is prepared by homogenizing at 65-70° C. glyceryl monostearate, triethyl citrate, TWEEN 80 and simethicone in distilled water. The GMS dispersion is then cooled to room temperature prior to the addition of the EUDRAGIT dispersion to form the final polymer dispersion. Separately, colorant (e.g., OPADRY II (white); Colorcon, West Point, Pa., USA) is mixed in distilled water to form the color dispersion.

The tablet cores to be coated were first placed in a perforated coating pan (e.g., Compu-Lab or Accela-Cota) and substantially coated with the final polymer dispersion mixture until all the final polymer dispersion mixture was consumed. The coated tablets are subsequently coated with the color dispersion mixture and the film-coated tablets were then allowed to cure in an oven at 40° C. for 24 hours.

### Example II

### Formula B

In a non-limiting formulation of the invention, Formula B, the following ingredients (Table IV) were used in the tablet core and in the film coat.

TABLE IV
Glipizide Sustained-release Tablets

| | | | | |
|---|---|---|---|---|
| | | Range % | Exam- | |
| Ingredient | Grade | (w/w) | ple % | Function |

Tablet Cores

| | | | | |
|---|---|---|---|---|
| Glipizide | | 1-5 | 2.5 | Active drug |
| Carbomer | 934P NF | 2-30 | 6.0 | Rate con- |
| | | | | trolling |
| | | | | polymer |
| Hydroxy- | Viscosity | 5-40 | 14.0 | Rate con- |
| propyl | 4,000-15,000 cps | | | trolling |
| Methyl- | | | | polymer |
| cellulose | | | | |
| Acacia | | 0-50 | 35.0 | Filler |
| | | | | (& binder) |
| Lactose | | 0-70 | 41.5 | Filler |
| Silicon | | 0.1-2 | 0.4 | Glidant |
| Dioxide | | | | |
| Magnesium | | 0.2-3 | 0.6 | Lubricant |
| Stearate | | | | |
| Total | | | 100.0 | |

Coat Formula

| | | | |
|---|---|---|---|
| Tablet Cores | 85-98 | 94.0 | |
| EUDRAGIT | 1-15 | 4.00 | Coating poly- |
| NE 30 | | | mer |
| EUDRAGIT | | | |
| L 30 D-55 | | | |
| Triethyl | 0.2-5 | 0.80 | Plasticizer |
| Citrate | | | |
| Glyceryl | 0.02-0.5 | 0.12 | Glidant |
| Monostea- | | | |
| rate | | | |
| TWEEN 80 | 0-0.1 | 0.025 | Surfactant |
| Simethicone | 0-0.01 | 0.005 | Antifoaming |
| | | | agent |
| Colorant | 0-5 | 1.05 | Colorant |
| (e.g. | | | |
| OPADRY II | | | |
| White) | | | |
| Total | | 100.00 | |

To prepare the tablet core of Formula B, all of the ingredients (excepting magnesium stearate) listed in Table IV were blended together in a PK blender. Fifty percent (50%) of the magnesium stearate was then added to the mixture, which was subsequently blended again. After the second blending, the blended mixture was compressed into slugs, which are then passed through a miller. The remaining magnesium stearate is added to the milled granules and blended again to form the final core blend. The milled final core blend was then compressed into tablets.

After formation, the tablet cores were next coated with a film coat having the formula described in Table IV. To coat the tablet core with the indicated amount of film coat, a EUDRAGIT NE 30D dispersion is added to a EUDRAGIT L 30D 55 dispersion to form the EUDRAGIT dispersion. Separately, a GMS dispersion is prepared by homogenizing at 65-70° C. glyceryl monostearate, triethyl citrate, TWEEN 80 and simethicone in distilled water. The GMS dispersion is then cooled to room temperature prior to the addition of the EUDRAGIT dispersion to form the final polymer dispersion. Separately, colorant is mixed in distilled water to form the color dispersion.

The tablet cores to be coated were first placed in a perforated coating pan (e.g., Compu-Lab or Accela-Cota) and substantially coated with the final polymer dispersion mixture until all the final polymer dispersion mixture was consumed. The coated tablets are subsequently coated with the color dispersion mixture and the film-coated tablets were then allowed to cure in an oven at 40° C. for 24 hours.

### Example III

### Formula C

In a non-limiting formulation of the invention, Formula C, the following ingredients were used in the tablet core and in the film coat (Table II, supra).

To prepare the tablet core of Formula C, all of the ingredients (excepting magnesium stearate) listed in Table II were blended together in a PK blender. Fifty percent (50%) of the magnesium stearate was then added to the mixture, which was subsequently blended again. After the second blending, the blended mixture was compressed into slugs, which are then passed through a miller. The remaining magnesium stearate is added to the milled granules and blended again to form the final core blend. The milled final core blend was then compressed into tablets. One of skill in the art will appreciate that alternative methods to assemble a tablet core are well known in the art and could be used instead of the steps described supra without altering the invention or its effectiveness.

After formation, the tablet cores were next coated with a film coat having the formula described in Table II.

To coat the tablet core with the indicated amount of film coat, a EUDRAGIT L 100 dispersion is prepared by dissolving the EUDRAGIT L 100 in isopropyl alcohol (may contain small amount of purified water) while stirring. Triethyl citrate is then added to the EUDRAGIT L 100 dispersion. The talc is separately dispersed in isopropyl alcohol, which is then added to the EUDRAGIT L 100 dispersion to form the final polymer dispersion.

The tablet cores to be coated were first placed in a perforated coating pan (e.g., Compu-Lab or Accela-Cota) and substantially coated with the final polymer dispersion mixture until all the final polymer dispersion mixture was consumed. The film-coated tablets were then allowed to cure in an oven 50° C. for 24 hours.

### Example IV

### Formula D

In a non-limiting formulation of the invention, Formula D, the following ingredients (Table I) were used in the tablet core and in the film coat. To prepare the tablet core of Formula D, all of the ingredients (excepting magnesium stearate) listed in Table I were blended together in a PK blender. Fifty percent (50%) of the magnesium stearate was then added to the mixture, which was subsequently blended again. After the second blending, the blended mixture was compressed into slugs, which are then passed through a miller. The remaining magnesium stearate is added to the milled granules and blended again to form the final core blend. The milled final blend was then compressed into tablets. After formation, the tablet cores were next coated with a film coat having the formula described in Table I.

To coat the tablet core with the indicated amount of film coat, an aqueous dispersion of ethylcellulose and triethyl citrate is prepared by stirring these ingredients (ethylcellulose, AQUACOAT ECD) and triethyl citrate) with water to form the final polymer dispersion. The tablet cores to be coated were first placed in a perforated coating pan (e.g., Compu-Lab or Accela-Cota) and substantially coated with the final polymer dispersion mixture until all of the final polymer dispersion was consumed. The film-coated tablets were then allowed to cure in an oven at 40° C. for 24 hours.

### Example V

### Formula E

In a non-limiting formulation of the invention, Formula E, the following ingredients were used in the tablet core (Formula C) and in the film coat (Table V, below).

TABLE V
Glipizide Sustained-release Tablets

| | | | | |
|---|---|---|---|---|
| | | Range % | Exam- | |
| Ingredient | Grade | (w/w) | ple % | Function |

Tablet Cores

| | | | | |
|---|---|---|---|---|
| Glipizide | | 1-5 | 2.5 | Active drug |
| Carbomer | 934P NF | 2-30 | 6.0 | Rate controlling |
| | | | | polymer |
| Hydroxy- | Viscosity | 5-40 | 14.0 | Rate controlling |
| propyl | 4,000-15,000 cps | | | polymer |
| Methyl- | | | | |
| cellulose | | | | |
| Acacia | | 0-50 | 35.0 | Filler (& binder) |
| Lactose | | 0-70 | 41.5 | Filler |
| Silicon | | 0.1-2 | 0.4 | Glidant |
| Dioxide | | | | |
| Magnesium | | 0.2-3 | 0.6 | Lubricant |
| Stearate | | | | |
| Total | | | 100.0 | |

Coat Formula

| | | | |
|---|---|---|---|
| Tablet Cores | 85-98 | 92.1 | |
| EUDRAGIT | 1-15 | 5.00 | Coating |
| L 30 D-55 | | | polymer |
| Triethyl | 0.1-5 | 0.60 | Plasticizer |
| Citrate | | | |
| Talc | 0-10 | 2.3 | Glidant |
| Total | | 100.00 | |

After formation of the tablet cores as in Example III above, the tablets were next coated with a film coat having the formulation described in Table V supra. The talc and triethyl citrate are dispersed in a small amount of distilled water which is then added to the EUDRAGIT L 30D-55 dispersion to form the final polymer dispersion.

The tablet cores to be coated were first placed in a perforated coating pan (e.g., Compu-Lab or Accela-Cota) and substantially coated with the final polymer dispersion mixture until all of the final polymer dispersion mixture was consumed. The film-coated tablets were allowed to cure in an oven at 40° C. for 24 hours.

### Example VI

### Release of Glipizide from Formula A

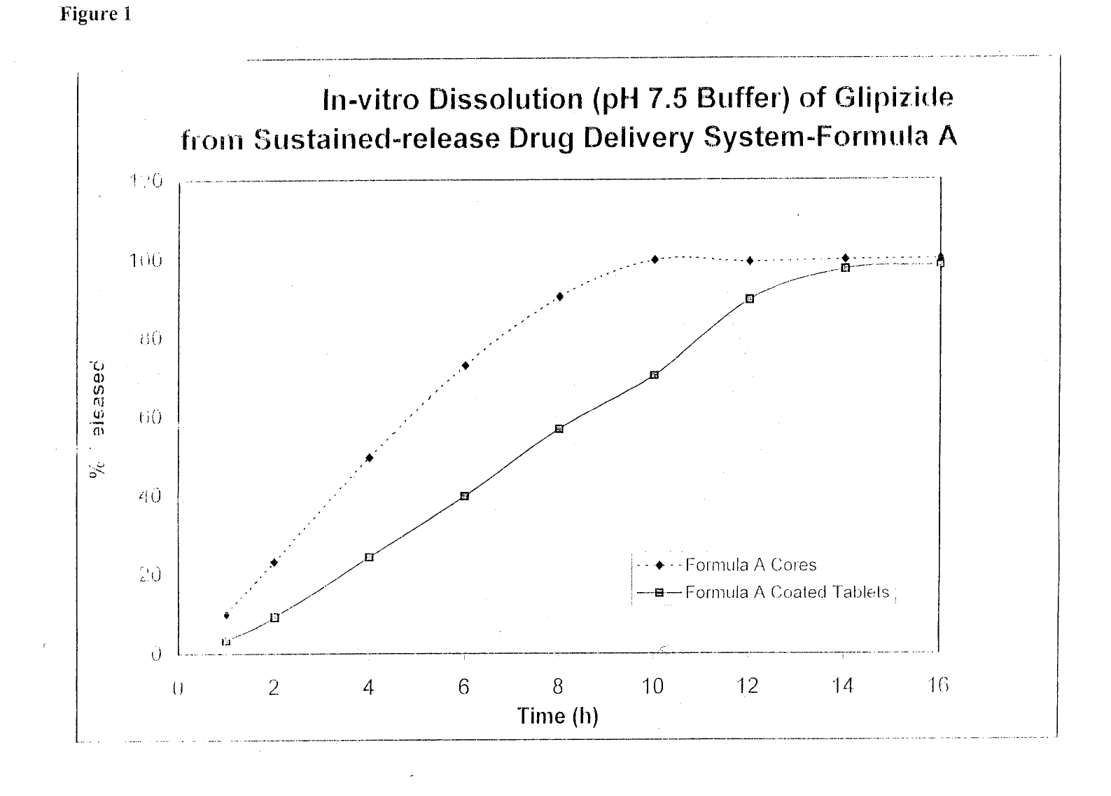

Formula A tablet cores and coated tablets (as described in Example I supra) were placed into phosphate buffered dissolution media having a pH of 7.5. The release of glipizide from the tablet cores or coated tablets was measured using a standard USP dissolution apparatus 2 and a calibrated HPLC method at 1, 2, 4, 6, 10, 12, 14 and 16 hours following placement of the tablet cores or coated tablets into the dissolution media. The results for glipizide release are represented schematically in FIG. 1. As FIG. 1 demonstrates, the Formula A tablet cores produce a sustained release of glipizide. When the Formula A tablet cores are coated (squares on FIG. 1), the release of glipizide after placement of the tablet into the pH 7.5 dissolution media occurs with no drug dumping effect.

The release behavior of glipizide from Formula A tablet cores or coated tablets was fitted to an exponential model introduced by Ritger and Peppas (J. Controlled Release 5: 37-42, 1987). This model is suited to polymeric swelling systems. This model is based on the equation: Mₜ/M_{T} =ktⁿ where t represents time, Mₜ/M_{T} is the fraction of drug release at time t, k is the kinetic constant, and n is the release exponent indicative of the mechanism of release.

To determine the “n” value for Formula A tablet cores and coated tablets, the logarithmic form of the above equation is employed wherein Log (Mₜ/M_{T})=Log k+n·Log t (data not shown).

### Example VII

### Release of Glipizide from Formula B

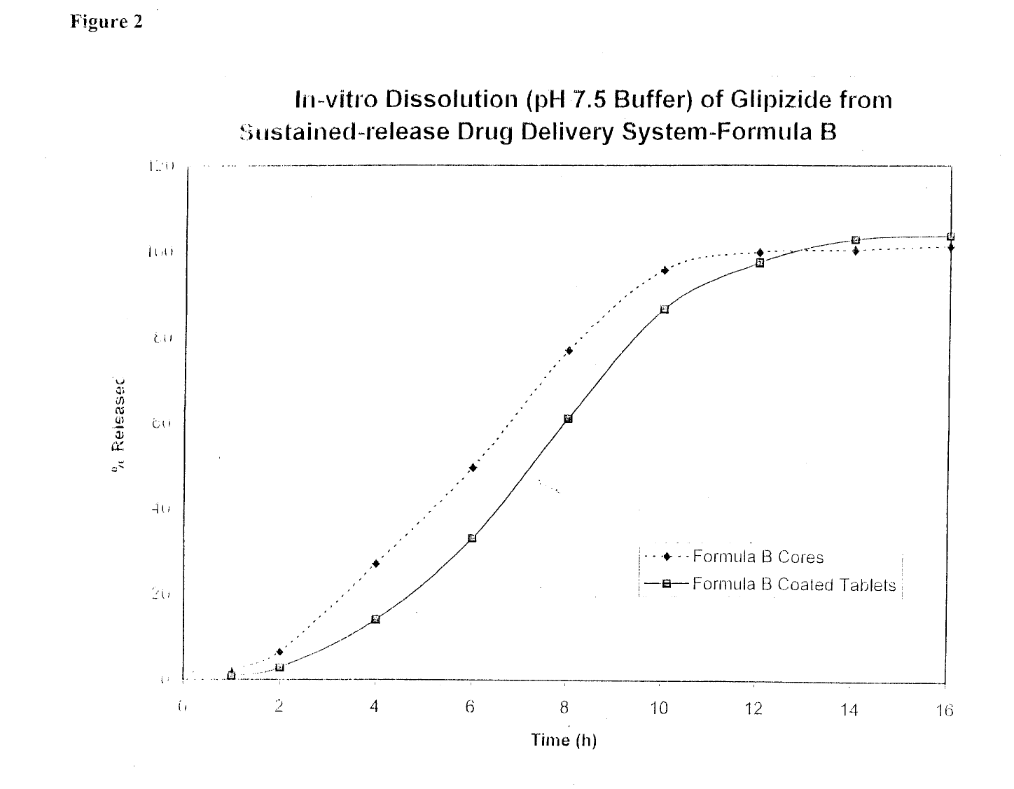

Formula B tablet cores and coated tablets (as described in Example II supra) were placed into phosphate buffered dissolution media having a pH of 7.5. The release of glipizide from the tablet cores or coated tablets was measured using a standard USP dissolution apparatus 2 and a calibrated HPLC method at 1, 2, 4, 6, 10, 12, 14 and 16 hours following placement of the tablet cores or coated tablets into the dissolution media. The results for glipizide release are represented schematically in FIG. 2. FIG. 2 demonstrates, that Formula B tablet cores produced a sustained release of glipizide without drug dumping.

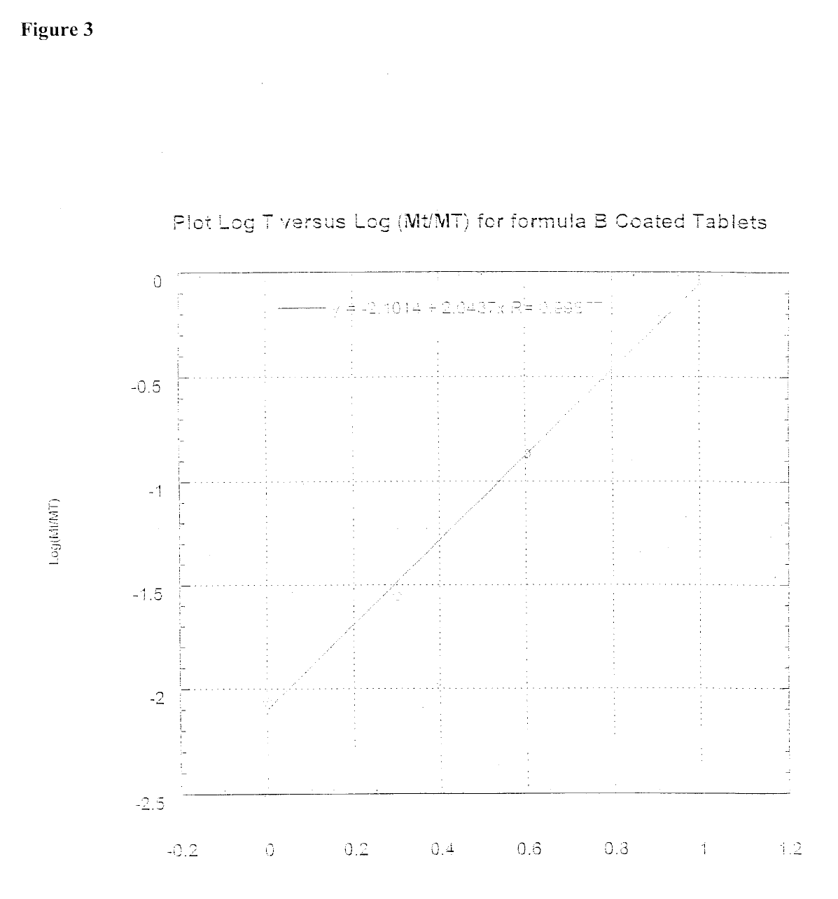

The data (≧70%) for dissolution was applied to the release equation Mₜ/M_{T}=ktⁿ (where t represents time, Mₜ/M_{T} is the fraction of drug release at time t, k is the kinetic constant, and n is the release exponent). The Log t and Log (Mₜ/M_{T}) values for Formula B were calculated and the values plotted graphically in FIG. 3. The kinetic constant k and exponent n values for Formula B tablet cores and coated tablets are shown in Table VI supra. As Table VI demonstrates, the value of n is about 1.71 for the tablet cores and about 2.04 for coated tablets. These results also indicate that the model of the drug release from Formula B glipizide tablets was non-Fickian super Case II type and the drug can be released at nearly zero-order rate through the matrix with a few hour lag time.

### Example VIII

### Release of Glipizide In Vivo

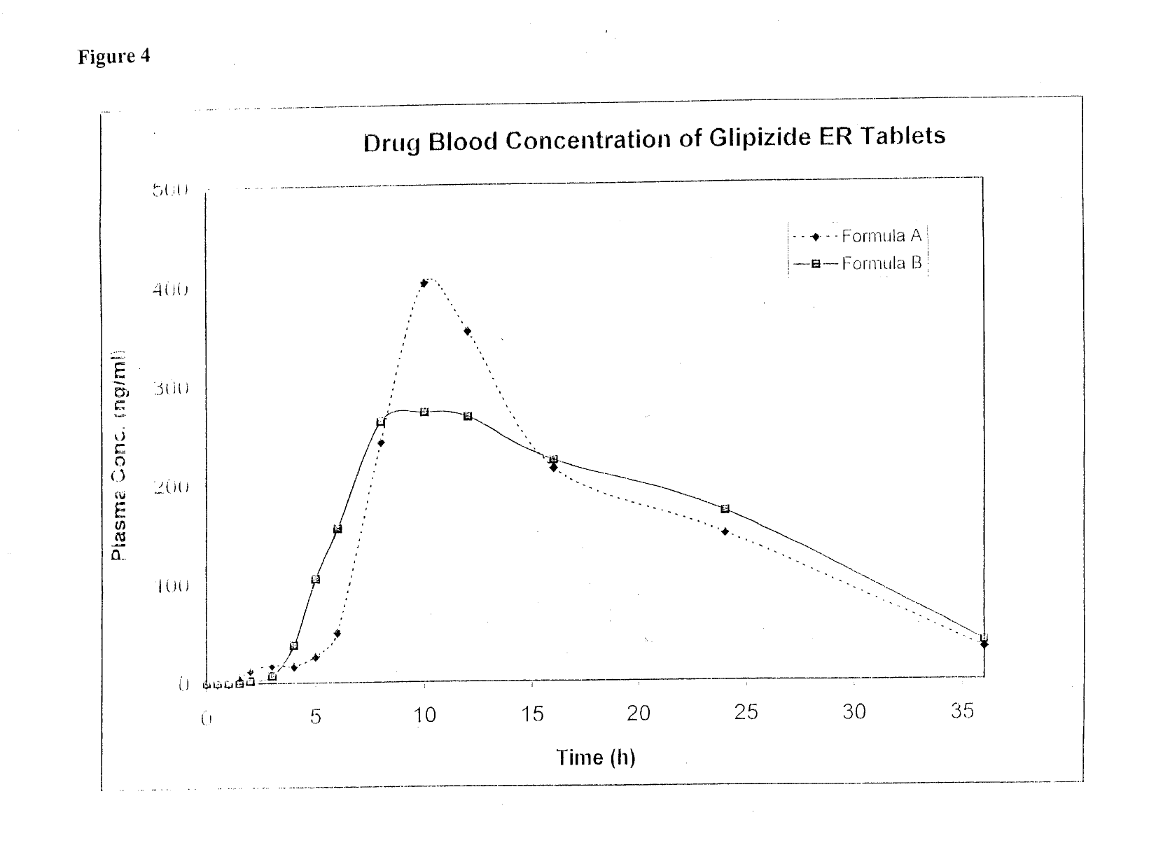

A single dose crossover clinical study was performed on 11 healthy volunteers. A single 10-mg glipizide tablet was ingested under a fasting condition. Blood samples were subsequently drawn at predetermined time intervals and the plasma level of drug determined by the specific, validated HPLC LC/MS method. The blood plasma levels for glipizide are graphically shown in FIG. 4 and the active drug substance was gradually released over 24 hours.

### Example IX

### Formula F

In a non-limiting example of the invention, Formula F, the following ingredients are used in the tablet core and in the film coat (Table VI, below).

TABLE VI
Glipizide Sustained-release Tablets

| | | | |
|---|---|---|---|
| | | Range % | |
| Ingredient | Grade | (w/w) | Function |

Tablet Cores

| | | | |
|---|---|---|---|
| Glipizide | | 1-5 | Active drug |
| Carbomer | 934P NF | 2-30 | Rate controlling |
| | | | polymer |
| Polyethylene Oxide | M.W. 2,000,000 | 5-40 | Rate controlling |
| | | | polymer |
| Acacia | | 0-50 | Filler (& binder) |
| Lactose | | 0-70 | Filler |
| Silicon Dioxide | | 0.1-2 | Glidant |
| Magnesium Stearate | | 0.2-3 | Lubricant |

Coat Formula

| | | |
|---|---|---|
| Tablet Cores | 85-98 | |
| EUDRAGIT L 30 | 1-15 | Coating polymer |
| D-55 | | |
| Triethyl Citrate | 0.1-5 | Plasticizer |
| Talc | 0-10 | Glidant |

After formation of the tablet cores as in Example I above, the tablets are next coated with a film coat having the formulation described in Table VI supra. The talc and triethyl citrate are dispersed in a small amount of distilled water which is then added to the EUDRAGIT L 30D-55 dispersion to form the final polymer dispersion.

The tablet cores to be coated are first placed in a perforated coating pan (e.g., Compu-Lab or Accela-Cota) and substantially coated with the final polymer dispersion mixture until all of the final polymer dispersion mixture was consumed. The film-coated tablets are allowed to cure in an oven at 40° C. for 24 hours.

### Equivalents

While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

What is claimed is:

1. A composition for the sustained delivery of glipizide, comprising glipizide and a carbomer admixed to form a core substantially coated with a film coat comprising a film coat polymer.

2. The composition of claim 1, comprising from about 0.1% to about 15% by weight of the core of glipizide, and from about 2% to about 30% by weight of the core of carbomer.

3. The composition of claim 1, comprising about 2.5% by weight of the core of glipizide, and about 6% by weight of the core of carbomer.

4. The composition of claim 1, wherein the core further comprises at least one core polymer chosen from the group consisting of hydroxypropyl methylcellulose and polyethylene oxide.

5. The composition of claim 4, comprising from about 5% to about 50% by weight of the core of hydroxypropyl methylcellulose.

6. The composition of claim 4, comprising about 31% by weight of the core of hydroxypropyl methylcellulose.

7. The composition of claim 4, comprising from about 14% to about 15% by weight of the core of hydroxypropyl methylcellulose.

8. The composition of claim 5, comprising from about 10% to about 30% by weight of the core of polyethylene oxide.

9. The composition of claim 5, comprising about 15% by weight of the core of polyethylene oxide.

10. The composition of claim 2, comprising about 15% by weight of the core of hydroxypropyl methylcellulose and about 15% by weight of the core of polyethylene oxide.

11. The composition of claim 1, wherein the film coat polymer comprises at least one pH-dependent or pH-independent polymer.

12. The composition of claim 11, wherein the pH-dependent polymer is chosen from the group consisting of methacrylic acid/methyl methacrylate copolymer, methacrylic acid/ethyl acrylate copolymer, methacrylic acid/methyl acrylate and methyl methacrylate copolymer, cellulose acetate phthalate, cellulose acetate trimellitate, hydroxypropyl methylcellulose acetate succinate, hydroxypropyl methylcellulose phthalate, polyvinyl acetate phthalate, methacrylic acid copolymer type C, methacrylic acid copolymer type A, polyacrylate dispersion, and shellac.

13. The composition of claim 11, wherein the pH-independent polymer is chosen from the group consisting of ethylcellulose, methacrylic ester copolymer, and ammonio methacrylate copolymer.

14. The composition of claim 11, comprising from about 1% to about 20% by weight of the core of a coat polymer.

15. The composition of claim 11, comprising from about 3% to about 5% by weight of the core of a coat polymer.

16. The composition of claim 11, comprising a total of about 3% by weight of the core of one or more film coat polymers chosen from the group consisting of methacrylic acid copolymer type A, methacrylic acid copolymer type C, ethylcellulose, and polyacrylate dispersion.

17. A composition for the sustained delivery of glipizide, comprising about 94% of a core and about 6% of a film coat by weight of the composition, wherein the core comprises about 2.5% of glipizide, about 15% of polyethylene oxide, and about 31% of hydroxypropyl methylcellulose by weight of the core, and the coat polymer comprises a total of about 4% of methacrylic acid/methyl methacrylate copolymer and methacrylic ester copolymer by weight of the composition.

18. A composition for the sustained delivery of glipizide, comprising about 94% of a core and about 6.0% of a film coat by weight of the composition, the core comprising about 2.5% of glipizide, about 14% of carbomer by weigh of the core, and the coat polymer comprising about 4% of methacrylic acid copolymers by weight of the composition.

19. A composition for the sustained delivery of glipizide, comprising about 95.7% of a core and about 4.3% of a film coat by weight of the composition, the core comprising about 2.5% of glipizide, about 14% of hydroxypropyl methylcellulose, and about 6% of carbomer by weight of the core, and the coat polymer comprising about 3% of methacrylic acid copolymer by weight of the composition.

20. A composition for the sustained delivery of glipizide, comprising about 96.3% of a core and about 3.7% of a film coat by weight of the composition, the core comprising about 2.5% of glipizide and about 15% of a hydroxypropyl methylcellulose by weight of the core, and the film coat comprising about 3% of ethyl cellulose by weight of the composition.

21. A composition for the sustained delivery of glipizide, comprising about 92.1% of a core and about 7.9% of a film coat by weight of the composition, the core comprising about 2.5% of glipizide, about 14% of hydroxypropyl methylcellulose, and about 6% of carbomer by weight of the core, and the film coat comprising about 5% of methacrylic acid copolymer by weight of the composition.
